(19) United States
(12) Patent Application Publication
(10) Pub. No.: US 2024/0422491 A1
(43) Pub. Date: Dec. 19, 2024

(54) SOUND SYSTEM FOR TREATING TINNITUS

(71) Applicant: Vitality Universe Sàrl, Grandson (CH)

(72) Inventor: Pier Rubesa, Grandson (CH)

(73) Assignee: Vitality Universe Sàrl, Grandson (CH)

(21) Appl. No.: 18/703,289

(22) PCT Filed: Oct. 19, 2022
(86) PCT No.: PCT/IB2022/060040

§ 371 Date: Apr. 19, 2024

(30) Foreign Application Priority Data
Oct. 19, 2021 (CH) 070418/2021

(51) Int. Cl. H04R 25/00; A61B 5/12; H04R 3/04; H04R 3/14

(52) U.S. Cl. CPC H04R 25/75; H04R 3/04; H04R 3/14; A61B 5/128

(57) ABSTRACT

The present invention relates to a sound system suitable for the treatment of tinnitus in a patient having an audio signal generator adapted to generate an audio signal based on a preset profile of a patient's tinnitus. The profile has at least one tinnitus frequency of the tinnitus A first device broadcasts an audio signal to the patient, the audio signal broadcasting device being connected to the audio signal generator. The audio signal has a fundamental frequency between 20 Hz and 300 Hz, preferably between 40 Hz and 160 Hz. At least one harmonic of the fundamental frequency, the harmonic being equal to the at least one tinnitus frequency plus or minus 10%, preferably to the at least one tinnitus frequency plus or minus 6%, and in that the first audio signal broadcasting device is adapted to broadcast the audio signal. The present invention also relates to a method of generating an audio signal on the basis of a tinnitus profile and of broadcasting this audio signal having a fundamental frequency of between 20 Hz and 300 Hz, preferably between 40 Hz and 160 Hz, and at least one harmonic of said fundamental frequency, the harmonic being equal to the at least one tinnitus frequency plus or minus 10%, preferably to the at least one tinnitus frequency plus or minus 6%.

### TECHNICAL AREA

The present invention relates to a sound system for generating and broadcasting audio signals suitable for treating tinnitus in a patient. The present invention also relates to a method for generating and broadcasting audio signals.

### BACKGROUND ART

Tinnitus is an auditory perception phenomenon in which a person perceives a sound whose origin is not external to their body. This sound is only perceived by the person affected by tinnitus, and remains inaudible to other people in the vicinity. The prevalence of this phenomenon in the adult population is between 10% and 20%, depending on studies and estimates, with a slight over-representation in the male population. Tinnitus is considered a symptom with various causes or contributing factors, such as
age
various types of sound trauma
injury to the hearing system
ear and sinus infections
heart or blood vessel disease
Meniere's disease
various brain tumors
hormonal changes, particularly in women
thyroid abnormalities
dental surgery
bruxism
stress
etc. . . .

In most cases, the phenomenon of tinnitus is essentially subjective, i.e. no vibration of any part of the human body can explain the sound heard by the person. The sound is often perceived as continuous, i.e. without interruption, although non-continuous sounds have also been documented. The intensity and frequency of tinnitus can also vary, particularly with increasing age.

Tinnitus is generally perceived identically in the left and right ears, although in rare cases asymmetrical perception between the two ears is possible. Tinnitus may be perceived as a pure tone, or as a more complex, colored sound with a dominant frequency.

Although subject to considerable variation between individuals, the human audible spectrum conventionally extends from 20 Hz to 20,000 Hz. The audible spectrum is generally divided into three main ranges
low frequencies: from 20 Hz to 300 Hz;
medium frequencies: from 300 Hz to 5′000 Hz;
High frequencies: from 5′000 Hz to 20′000 Hz;
   The average frequency of tinnitus in the population is around 5,000 Hz, while the majority of tinnitus is between 500 Hz and 10,000 Hz.

Apart from the auditory system, many parts of the human body are usually capable of perceiving certain acoustic vibrations, particularly those produced by low-frequency sound waves. For example, the human epidermis, body hair and skeleton are equipped with sensors capable of sensing certain low-frequency sound vibrations.

Low-frequency vibrations are thus detected not only by the ear, but also by other parts of the body, leading to a combined signal that is analyzed differently by the brain than higher frequencies. In short, medium and high frequencies are usually detected and perceived by the ear and auditory cortex directly, while certain low frequencies are perceived not only by these organs, but also by the somatosensory cortex through the whole body's nervous system. Tinnitus has been shown to involve brain areas beyond those linked to the auditory system, i.e. beyond the auditory cortex. These areas can, however, be stimulated via the nervous system, notably by low-frequency sound vibrations.

Numerous methods have been developed to provide temporary relief for tinnitus sufferers, or to gradually acclimatize them to the condition. While these methods may help people to relax or live better with the symptom, none of them has succeeded in treating and eliminating the symptom in a reproducible way. Most of these methods are based on the principle of masking, i.e. the construction of sounds that temporarily mask the patient's perception of tinnitus in order to induce relaxation. To ensure that the perception of these sounds is pleasant and/or effective, these methods are generally based on sounds with an “easily or pleasantly audible” average frequency, i.e. between 1000 Hz and 10,000 Hz, or even a more restricted range in the case of a hearing deficit in the patient.

U.S. Pat. No. 6,682,472 B1 describes a relaxation method and device for a person suffering from tinnitus. The method consists in modifying the audio spectrum of a signal on the basis of a predetermined masking algorithm modifying the intensity of the signal at certain frequencies. The masking algorithm enables temporary masking of the tinnitus (during signal peaks) and occasional perception of the tinnitus (during signal troughs). This masking produces a feeling of relief and relaxation in the person affected by the tinnitus.

WO 2020128053 A2 describes a device comprising an identification unit of at least one tinnitus parameter, an adaptation unit configured to modify a received audio signal or to generate the audio signal based on the identified parameter. A defined frequency spectrum of the audio signal is based on a tinnitus suppression frequency. The device further comprises an audio output unit for broadcasting the audio signal to the user. The frequency ranges disclosed in this document for the construction of the audio signal are relatively high, between 1000 Hz and 10′000 Hz, i.e. located in the medium or high frequency range. Moreover, only the auditory system (inner, middle and outer ear) is involved.

### BRIEF SUMMARY OF THE INVENTION

One aim of the present invention is to propose a sound system suitable for the treatment of tinnitus free from the limitations of known systems.

Another aim of the invention is to propose a sound system suitable for the treatment of tinnitus involving a wider range of body sensors to induce an improved sense of relaxation in the patient.

Another aim of the invention is to propose a sound system suitable for the treatment of tinnitus that stimulates brain areas not related to the auditory system.

Another aim of the invention is to propose a method for generating and broadcasting sounds that induce increased relaxation in a patient suffering from tinnitus.

According to the invention, these aims are achieved in particular by means of a sound system adapted for the treatment of tinnitus in a patient comprising
an audio signal generator adapted to generate an audio signal based on a preset profile of a patient's tinnitus, said profile comprising at least one tinnitus frequency of said tinnitus;
a first audio signal broadcasting device to the patient, the first audio signal broadcasting device being connected to the audio signal generator;
   characterized in that said audio signal comprises:
a fundamental frequency between 20 Hz and 300 Hz, preferably between 40 Hz and 160 Hz, and;
at least one harmonic of said fundamental frequency, said harmonic being equal to the at least one tinnitus frequency plus or minus 10%, preferably to the at least one tinnitus frequency plus or minus 6%,
   and in that said first audio signal broadcasting device is adapted to broadcast said audio signal.

The audio signal broadcasting device can include a subwoofer for broadcasting to the patient a low-frequency audio signal having a frequency equal to the fundamental frequency.

Advantageously, the subwoofer is placed in the direct vicinity of the patient to stimulate certain areas of the patient's body by the air vibration produced by the subwoofer.

The system can further comprise a patient support, the subwoofer being arranged so that vibrations corresponding to said fundamental frequency emitted by the subwoofer are propagated in the patient support.

The patient support may consist, for example, of a bed, a massage table or a seat.

The system may further comprise a second audio signal broadcasting device adapted to broadcast a plurality of audio signals so as to objectify said tinnitus and generate the tinnitus profile.

The patient's tinnitus profile may include, in addition to tinnitus frequency, the following characteristics:
a tinnitus behavior measuring the discrete or continuous nature of the patient's tinnitus, and/or;
a tinnitus intensity measuring the volume (in decibels) at which the tinnitus is perceived by the patient, and/or;
a nature of the tinnitus sound, i.e. how the sound is perceived by the patient (the sound is perceived as warm, cold, coloured, etc.)

The first audio signal broadcasting device may comprise at least three loudspeakers.

The first audio signal broadcasting device may comprise headphones or a hearing aid for broadcasting the audio signal to the patient.

The system can also include a sound-isolated booth, with the audio signal broadcasting device located inside the booth.

The system can further comprise a database of a plurality of patient tinnitus profiles, the audio signal generator being connected to said database.

The above-mentioned aims are also achieved by means of a method of generating and broadcasting an audio signal comprising the steps of:
establishing a profile of a patient's tinnitus, said profile comprising at least one tinnitus frequency;
generating an audio signal based on the tinnitus profile, the audio signal comprising
a fundamental frequency between 20 Hz and 300 Hz, preferably between 40 Hz and 160 Hz, and;
   at least one harmonic of said fundamental frequency, said harmonic being equal to the at least one tinnitus frequency plus or minus 10%, preferably to the at least one tinnitus frequency plus or minus 6%;
broadcasting the audio signal using a first audio signal broadcasting device;

The tinnitus profile established in the first step may further include:
a tinnitus behavior measuring the discrete or continuous nature of the patient's tinnitus, and/or;
a tinnitus intensity measuring the volume (in decibels) at which the tinnitus is perceived by the patient, and/or;
a nature of the tinnitus sound, i.e. how the sound is perceived by the patient (the sound is perceived as warm, cold, coloured, etc.)

The tinnitus profiling step may comprise a phase of broadcasting a plurality of sounds to said patient by means of a second audio signal broadcasting device, and a phase of receiving feedback from the patient, said feedback being based on the patient's perception of the plurality of audio signals.

### BRIEF DESCRIPTION OF FIGURES

Examples of implementation of the invention are shown in the description illustrated by the appended figures in which:

FIG. 1 illustrates the audible spectrum.

FIG. 2 schematically illustrates a sound system suitable for treating tinnitus.

FIG. 3 illustrates the establishment of a patient's tinnitus profile.

FIG. 4 schematically illustrates a sound system comprising three loudspeakers and a subwoofer for broadcasting an audio signal to a patient.

FIG. 5 schematically illustrates a sound system comprising three loudspeakers and a subwoofer for broadcasting an audio signal to a patient in a sound-isolated booth.

### EXAMPLE(S) OF EMBODIMENT OF THE INVENTION

As illustrated in FIG. 1, the human audible spectrum conventionally extends from 20 Hz to 20,000 Hz. As explained above, this interval is generally divided into three ranges: low frequencies BF, between 20 Hz and 300 Hz; mid frequencies MF, between 300 Hz and 5′000 Hz; and high frequencies HF, between 5′000 Hz and 20′000 Hz. The average frequency F_{moy} of a tinnitus is around 5,000 Hz, while the majority of tinnitus has a medium or high frequency, between 500 Hz and 10,000 Hz.

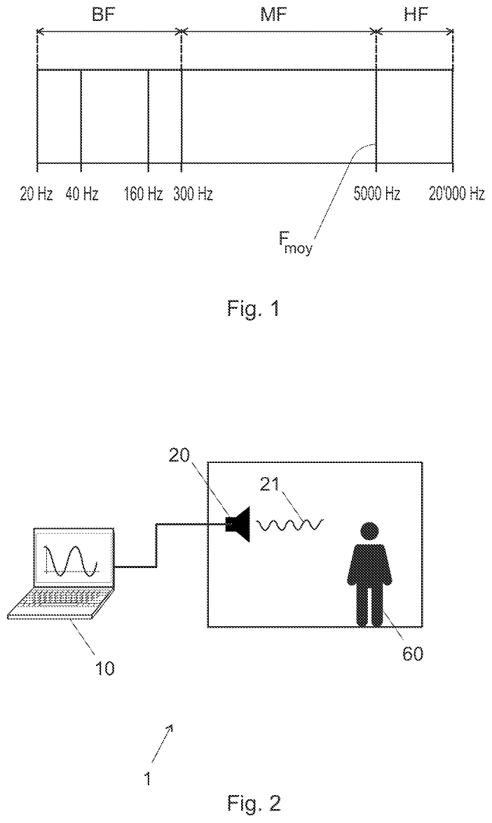

FIG. 2 schematically illustrates a sound system 1 adapted for the treatment of tinnitus comprising an audio signal generator 10 generating an audio signal and a first sound diffusion device 20 for diffusing this audio signal 21 to a patient 60.

The audio signal 21 is generated based on a previously established patient's tinnitus profile, which includes at least one patient tinnitus frequency. This patient tinnitus frequency is a theoretical frequency obtained from an objectification of the tinnitus perceived by the patient, so as to approximate the sound characteristics of the perceived tinnitus as accurately as possible.

In the context of the present invention, a wave generator 10 designates a device capable of generating a digital or analogue audio signal having the characteristics of an audible sound wave, in particular one or more frequencies between 20 Hz and 20′000 Hz. It can therefore be a processor causing the execution of a software allowing the generation of such an audio signal, and, in a preferred embodiment, consist of a computer such as a PC equipped with a specific audio signal generation program. In another embodiment, the wave generator 10 comprises a specifically dedicated device such as a tone generator or other types of special signal generator.

In particular, the wave generator 10 is adapted to generate an audio signal having a fundamental frequency of between 20 Hz and 300 Hz, preferably between 40 Hz and 160 Hz, and at least one harmonic of this fundamental frequency equal to the tinnitus frequency plus or minus 10%, preferably the tinnitus frequency plus or minus 6%. Harmonics are frequencies obtained by multiplying a given frequency, in this case the fundamental frequency, by an integer.

In a particular embodiment, a tinnitus profile of a patient 60 comprises a tinnitus frequency equal to 5000 Hz. Starting from this tinnitus frequency of 5000 Hz, a fundamental frequency is determined, for example by descending a finite number of octaves until the value lies between 40 Hz and 160 Hz. As descending an octave corresponds to dividing the frequency by two, descending 5 octaves from the 5000 Hz value gives a fundamental frequency equal to 5000 Hz/2⁵=156.25 Hz. Alternatively, a fundamental frequency equal to 5000 Hz/2⁶=78.125 Hz can be set.

For a starting tinnitus frequency equal to 7,000 Hz, it is necessary to descend 6 octaves to obtain a fundamental frequency between 40 Hz and 160 Hz since 7000 Hz/2⁶=109.375 Hz, whereas for a starting tinnitus frequency, only a 2 octave descent is necessary to obtain a fundamental frequency between 40 Hz and 160 Hz since 500 Hz/2²=125 Hz.

As mentioned above, the audio signal produced by the wave generator 10 comprises one or more harmonics of this fundamental frequency, this or these harmonics being equal to the tinnitus frequency plus or minus 10%, preferably plus or minus 6%. For a tinnitus frequency equal to 5000 Hz, the harmonics of the fundamental frequency therefore have harmonic frequencies between 4500 Hz and 5500 Hz, preferably between 4700 Hz and 5300 Hz. So, if the fundamental frequency is set equal to 156.25 Hz and a tolerance of 10% around the tinnitus frequency is allowed, the possible harmonics are

$$\begin{matrix}- 29 \times 156.25 Hz = 4531.25 Hz \\ - 30 \times 156.25 Hz = 4687.5 Hz \\ - 31 \times 156.25 Hz = 4843.75 Hz \\ - 32 \times 156.25 Hz = 5000 Hz , i . e . the tinnitus frequency \\ - 33 \times 156.25 Hz = 5156.25 Hz \\ - 34 \times 156.25 Hz = 5312.5 Hz \\ - 35 \times 156.25 Hz = 5468.75 Hz\end{matrix}$$

Whereas if a tolerance of 6% is allowed, the possible harmonics are 4843.75 Hz, 5000 Hz and/or 5156.25 Hz.

In a particular embodiment, the audio signal produced by the audio signal generator 10 comprises the fundamental frequency, a first harmonic of the fundamental frequency equal to the tinnitus frequency and at least one second harmonic of the fundamental frequency within the range of values of the tinnitus frequency plus or minus 10%, preferably plus or minus 6%.

In another embodiment, the audio signal comprises, in addition to the first and at least one second harmonic, at least one third harmonic of the fundamental frequency.

Once produced by the audio signal generator 10, the audio signal is then broadcasted by the first audio signal broadcasting device 20 connected to the audio signal generator. The audio signal broadcasting device can be adapted to convert a digital signal into an analog signal in the event that the audio signal generator produces a digital signal. Generally speaking, the audio signal broadcasting device produces a sound wave audible to the patient 60, this sound wave having physical characteristics (frequency, amplitude, etc.) corresponding to the characteristics of the audio signal.

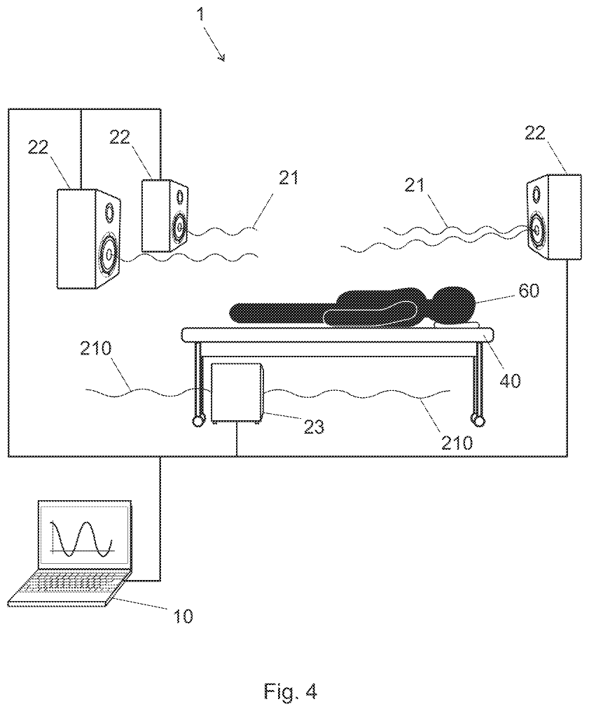

In a preferred embodiment illustrated in FIG. 4, the first audio signal broadcasting device comprises a plurality of loudspeakers 22 adapted to broadcast the audio signal described above. The first audio signal broadcasting device may also comprise a subwoofer 23 adapted to broadcast a low-frequency audio signal 210 comprising the fundamental frequency.

The loudspeakers 22 can be directional loudspeakers arranged so as to target a part of the patient's body in order to stimulate certain areas in particular thanks to the vibration of the sound wave corresponding to the audio signal broadcasted by the loudspeakers.

In the embodiment shown in FIG. 4, three directional speakers 22 and a subwoofer 23 are arranged around the patient 60. Two directional speakers are directed at the patient's legs, a first one at the left leg and a second one at the right leg. The third directional speaker is directed at the patient's head. Preferably, the subwoofer 23 is arranged so as to also stimulate certain areas of the patient's body by propagation of sound waves corresponding to the low-frequency signal 210 broadcasted by the subwoofer.

A greater or lesser number of speakers and/or subwoofers can of course be envisaged without departing from the scope of the present invention.

The loudspeakers 22 are adapted to broadcast the audio signal described above. In one embodiment, all the loudspeakers broadcast the same audio signal, while in an alternative embodiment, at least two loudspeakers each broadcast a different audio signal. For example, a first loudspeaker may broadcast an audio signal comprising a fundamental frequency between 40 Hz and 160 Hz and a first harmonic of the fundamental frequency equal to the tinnitus frequency, while a second loudspeaker broadcasts an audio signal comprising the same fundamental frequency and a second harmonic of the fundamental frequency within the range of values of the tinnitus frequency plus or minus 10%, preferably plus or minus 6%.

In an alternative embodiment, the first broadcasting device 20 comprises a listening device such as headphones or a hearing aid. In this embodiment, the audio signal generator 10 may comprise a computer program run by a web application or a mobile application so that the sound system 1 can be used by the patient himself, for example at home.

Tinnitus is mostly perceived as a continuous sound, unlike most of the sounds we perceive in our environment (e.g. voices, city and nature noises). However, the five human senses become accustomed to (or saturated by) continuous signals relatively quickly. For example, the touch sensors in the buttocks easily become accustomed to the contact of a seat (very quickly we no longer “feel” the seat's presence). Similarly, our eyes quickly become accustomed to the presence of colored glasses.

With regard to hearing, a great deal of research has investigated accommodation to a continuous audio signal in the short and long term. While short-term accommodation is generally located in the primary auditory cortex, long-term accommodation is more likely to be found in the lateral amygdala and cortical areas.

Thus, in order to involve this long-term accommodation process, the audio signal can be generated so as to be continuously audible for a sufficiently long determined duration. In one embodiment, the audio signal has a duration of at least three minutes and its broadcast is iterated at least 5 times, preferably at least 10 times. Alternatively or additionally, a succession of three-minute audio signals are broadcasted, each with a specific volume and frequency composition.

In order to maximize the stimulation of parts of the patient's body 60, the present system 1 may comprise a patient support 40 adapted to, on the one hand, support all or part of the patient's body, and on the other hand to propagate the air vibrations produced by the subwoofer 23 when broadcasting the low-frequency audio signal 210.

This support 40 can be a massage table, a bed or a seat, for example. Preferably, the patient support is adapted so that the patient's legs are in direct contact with the support when the patient is in the treatment position, i.e., when the audio signal, and in particular the fundamental frequency, is broadcast by the first audio signal broadcasting device.

In a preferred embodiment shown in FIG. 4, the patient support 40 consists of a massage table under which the subwoofer 23 is arranged. The massage table is oriented so that the subwoofer 23 is located under the end of the massage table adapted to support the patient's legs. This arrangement enables the sound vibrations emitted by the subwoofer to be transmitted to the massage table and thus to the parts of the patient's body in contact with the table, in particular his or her legs.

As mentioned above, a patient's tinnitus profile is established in order to determine characteristics of the patient's tinnitus, including its frequency. In addition to tinnitus frequency, these characteristics may include
a second tinnitus frequency in the case of dissociated perception between the right and left auditory systems;
a tinnitus behavior corresponding to the continuous or non-continuous nature of the tinnitus, i.e. whether the tinnitus manifests itself as a continuous sound or as a succession of sounds interspersed with silences;
a tinnitus intensity typically corresponding to the sound volume in decibels perceived by the patient;
the nature of the tinnitus sound, i.e. an indication of how the sound is experienced by the patient as warm/cold, colored, etc. . . .

In general, the tinnitus profile can contain all the physical characteristics of the sound perceived by the patient, so that the tinnitus can be objectified. These characteristics can then be used to create the audio signal generated and broadcasted by the system 1.

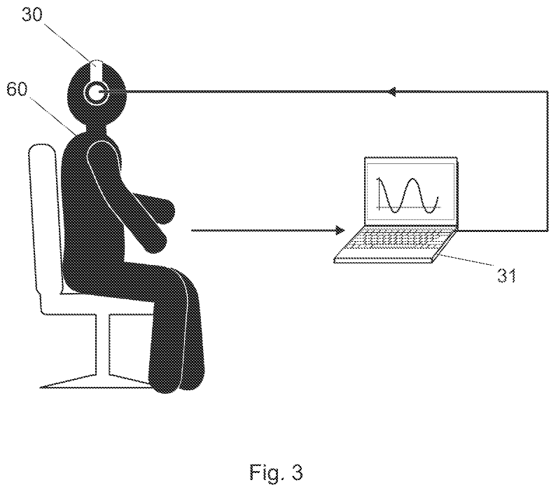

The tinnitus profile is obtained by broadcasting a plurality of sounds to the patient by means of a second audio signal broadcasting device 30, and by allowing the patient to give feedback on the sounds broadcasted to him. As illustrated in FIG. 3, the second audio signal broadcasting device 30 may comprise headphones connected to a computer 31. A computer program, whose execution is automatic or directed by an operator, controls the broadcasting of a plurality of sounds into the headphones. After listening to a sound, the patient gives feedback to the computer program, for example by alerting the operator verbally or by interacting directly with the computer program via an application. This feedback is then taken into account by the computer program or the operator to determine which sound to play next, or to establish one of the characteristics of the tinnitus profile.

A typical tinnitus establishment method, for example, involves playing a first sound to patient 60 through headphones. The patient then indicates whether the frequency he hears through the headphones is higher or lower than the frequency of his tinnitus. Based on this feedback, an operator then chooses to play a sound with a lower or higher frequency, iteratively to obtain a good approximation of the patient's tinnitus frequency. A similar methodology can be used for the other tinnitus characteristics mentioned above.

In a particular embodiment, tinnitus profiling is carried out using the sound system 1, in particular, the second audio signal broadcasting device 30 may be the same as the first broadcasting device 20, and the audio signal generator 10 is another computer program run by the same computer as that used for tinnitus profiling.

In an alternative embodiment, tinnitus profiling is carried out without operator intervention, using headphones and an enabling web or mobile application. In this case, the computer program is run automatically and the patient gives feedback to the computer program via the web or mobile application. The tinnitus profile thus established can then be stored electronically on an external medium or on a dematerialized medium such as a cloud in order to be used to generate an audio signal as explained above.

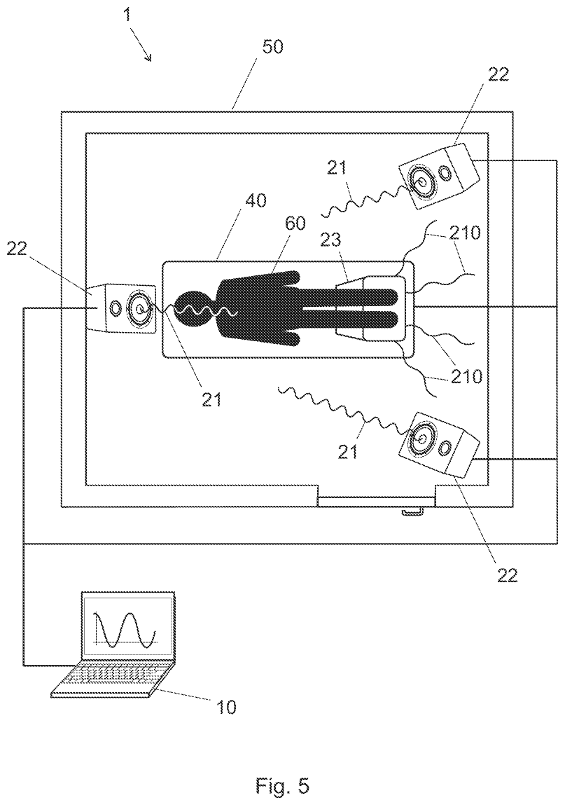

In an embodiment illustrated in FIG. 5, the sound system 1 further comprises a sound-isolated booth 50, inside which the first audio signal broadcasting system 20 and the patient support 40 supporting the patient 60 are arranged. The audio signal generator is located outside the sound-isolated booth, so that it can be controlled by an operator without sound interference with the patient.

According to an embodiment not shown, the sound system 1 further comprises a database of a plurality of tinnitus profiles of several patients. Such a database can, for example, be used by the audio signal generator 10 to produce the audio signal not only on the basis of a personalized tinnitus profile, but also on the basis of statistical elements determined from the plurality of tinnitus profiles stored in the database.

The database can be stored locally, for example on a computer containing the audio signal generator when the latter is a computer program running on a computer. Alternatively or additionally, the database can be stored dematerialized on a cloud or remote server.

The present invention also relates to a method for generating and broadcasting an audio signal. This method comprises the following steps:
establishing a profile of a patient's tinnitus, said profile including at least one tinnitus frequency;
generating an audio signal based on the tinnitus profile, the audio signal comprising
   a fundamental frequency between 20 Hz and 300 Hz, preferably between 40 Hz and 160 Hz, and;
   at least one harmonic of said fundamental frequency, said harmonic being equal to the at least one tinnitus frequency plus or minus 10%, preferably to the at least one tinnitus frequency plus or minus 6%;
broadcasting the audio signal using a first audio signal broadcasting device.

As already mentioned and explained above, the patient's tinnitus profile may include, in addition to a tinnitus frequency, one or more of the following characteristics:
a second tinnitus frequency;
a tinnitus behavior;
a tinnitus intensity;
a nature of the tinnitus sound;
any other physical characteristics of the sound perceived by the patient, so as to objectify the tinnitus.

As detailed above, this tinnitus profiling step may comprise a phase of broadcasting a plurality of sounds to said patient by means of a second audio signal broadcasting device, and a phase of receiving feedback from the patient, said feedback being based on the patient's perception of the plurality of audio signals.

### REFERENCE NUMERALS

1 Sound system for tinnitus treatment
10 Audio signal generator
20 First audio signal broadcasting device
21 Audio signal
210 Low-frequency audio signal
211 Fundamental frequency harmonics
22 Loudspeaker
23 Subwoofer
30 Second audio signal broadcasting device
31 Computer
40 Patient support
50 Sound-isolated booth
60 Patient
BF Low-frequency sound waves
MF Mid-frequency sound waves
HF High-frequency sound waves
F_{moy} Average tinnitus frequency

1. A sound system adapted for the treatment of tinnitus in a patient comprising
an audio signal generator adapted to generate an audio signal based on a preset profile of a patient's tinnitus, said profile comprising at least one tinnitus frequency of said tinnitus:
a first audio signal broadcasting device to the patient, the first audio signal broadcasting device being connected to the audio signal generator;
wherein said audio signal comprises:
a fundamental frequency between 20 Hz and 300 Hz, preferably between 40 Hz and 160 Hz, and;
at least one harmonic of said fundamental frequency, said harmonic being equal to the at least one tinnitus frequency plus or minus 10%, preferably to the at least one tinnitus frequency plus or minus 6%,
and in that said first audio signal broadcasting device is adapted to broadcast said audio signal.

2. The system according to claim 19, wherein the first audio signal broadcasting device comprises a subwoofer for broadcasting to the patient a low-frequency audio signal having a frequency equal to the fundamental frequency.

3. The system according to claim 2, wherein the system further comprises a patient support, the subwoofer being arranged so that vibrations corresponding to said fundamental frequency emitted by the subwoofer are propagated in the patient support.

4. The system according to claim 3, wherein the patient support is selected from: a bed, a massage table or a seat.

5. The system according to claim 1, wherein the system further comprises a second audio signal broadcasting device adapted to broadcast a plurality of audio signals so as to objectify said tinnitus and generate said tinnitus profile.

6. The system according to claim 1, wherein the patient's tinnitus profile further comprises:
a tinnitus behavior and/or:
a tinnitus intensity and/or;
a tinnitus sound nature.

7. The system according to claim 1, wherein the first audio signal broadcasting device comprises at least three loudspeakers.

8. The system according to claim 1, wherein the first audio signal broadcasting device comprises headphones or a hearing aid for broadcasting the audio signal to the patient.

9. The system according to claim 1, wherein the system further comprises a sound-isolated booth, the first audio signal broadcasting device being disposed inside the booth.

10. The system according to claim 1, wherein the system further comprises a database of a plurality of patient tinnitus profiles, the audio signal generator being connected to said database.

11. A method of generating and broadcasting an audio signal comprising the steps of:
establishing a profile of a patient's tinnitus, said profile comprising at least one tinnitus frequency;
generating the audio signal based on the tinnitus profile, the audio signal comprising
a fundamental frequency between 20 Hz and 300 Hz, preferably between 40 Hz and 160 Hz, and:
at least one harmonic of said fundamental frequency, said harmonic being equal to the at least one tinnitus frequency plus or minus 10%, preferably to the at least one tinnitus frequency plus or minus 6%;
broadcasting the audio signal using a first audio signal broadcasting device;

12. The method according to claim 11, wherein the tinnitus profile further comprises:
a tinnitus behavior and/or;
a tinnitus intensity and/or;
a tinnitus nature sound.

13. The method according to claim 11, wherein the step of establishing a tinnitus profile comprises a phase of broadcasting a plurality of sounds to said patient by means of a second audio signal broadcasting device, and a phase of receiving feedback from the patient, said feedback being based on the patient's perception of the plurality of audio signals.
